(19) United States
(12) Patent Application Publication
(10) Pub. No.: US 2023/0001143 A1
(43) Pub. Date: Jan. 5, 2023

(54) Vascular Access System Having a Dynamically Expandable Probe

(71) Applicant: Becton, Dickinson and Company, Franklin Lakes, NJ (US)

(72) Inventor: Jonathan Karl Burkholz, Salt Lake City, UT (US)

(21) Appl. No.: 17/852,489

(22) Filed: Jun. 29, 2022

Related U.S. Application Data
(60) Provisional application No. 63/218,140, filed on Jul. 2, 2021.

(51) Int. Cl. A61M 25/00

(52) U.S. Cl. CPC A61M 25/0082; A61M 2025/0019

(57) ABSTRACT

A vascular access system can have a probe with a dynamically expandable distal end. The dynamically expandable probe can be inserted through a catheter while the catheter is positioned intravenously to facilitate fluid flow into or out from the catheter such as by moving an occlusion away from the catheter tip, removing an occlusion, accessing additional sources of blood and/or repositioning the catheter tip. Because it is dynamically expandable, the probe can minimize the likelihood that it will become occluded while it is inserted intravenously and may provide control over the rate of fluid flow into or out from the catheter.

### CROSS-REFERENCE TO RELATED APPLICATION

The present application claims priority to U.S. Provisional Application Ser. No. 63/218,140, entitled “Vascular Access System Having a Dynamically Expandable Probe”, filed Jul. 2, 2021, the entire disclosure of which is hereby incorporated by reference in its entirety.

### BACKGROUND OF THE INVENTION

Vascular access systems are commonly used for a variety of infusion therapies. For example, a vascular access system may be used for infusing fluids, such as normal saline solution, various medicaments, and total parenteral nutrition, into a patient. Vascular access systems may also be used for withdrawing blood from the patient.

A common type of vascular access system is an over-the-needle peripheral intravenous (“IV”) catheter (“PIVC”). As its name implies, the over-the-needle catheter may be mounted over a needle having a sharp distal tip. The catheter and the needle may be assembled so that the distal tip of the needle extends beyond the distal tip of the catheter with the bevel of the needle facing up away from skin of the patient. The catheter and needle are generally inserted at a shallow angle through the skin into the vasculature of the patient. Once the catheter is positioned within the vasculature, it may become occluded such as when a thrombus forms around the catheter's distal opening or the distal opening is positioned against a vessel wall.

When vascular access systems are not adequately maintained within the patient's vasculature, they are likely to become occluded. Once a vascular access system is occluded, it may no longer be possible to use the vascular access system to infuse fluids or withdraw blood. In such cases, the vascular access system may be replaced. Yet, replacing a vascular access system is burdensome for the patient and increases costs. To address such issues, some instruments have been developed that can be inserted through the indwelling catheter of the vascular access system to remove the occlusion. For example, some devices employ rigid tubing that can be inserted through the catheter and distally beyond the catheter's distal opening. With the rigid tubing inserted in this manner, such devices can obtain a blood sample through the rigid tubing even if the catheter had become occluded. In other words, the rigid tubing is employed to physically pass through any occlusion that may have formed in or around the catheter's distal opening and forms a separate fluid pathway from the catheter for collecting the blood sample. However, the single opening of the tubing may become occluded with thrombus as it is pushed through and beyond the occlusion.

The subject matter claimed herein is not limited to embodiments that solve any disadvantages or that operate only in environments such as those described above. Rather, this background is only provided to illustrate one example technology area where some implementations described herein may be practiced.

### SUMMARY OF THE INVENTION

The present disclosure relates generally to vascular access systems having a dynamically expandable probe. The dynamically expandable probe can be inserted through a catheter while the catheter is positioned intravenously to facilitate fluid flow into or out from the catheter such as by moving an occlusion away from the catheter tip, removing an occlusion, accessing additional sources of blood and/or repositioning the catheter tip. Because it is dynamically expandable, the probe can minimize the likelihood that it will become occluded while it is inserted intravenously and may provide control over the rate of fluid flow into or out from the catheter.

In some embodiments of the present disclosure, a probe assembly may include a probe housing, a probe that extends within the probe housing and that has a dynamically expandable distal end and a probe actuator that is configured to advance the probe from a proximal position to a distal position. The probe actuator may also be configured to expand the dynamically expandable distal end.

In some embodiments, the probe comprises a core segment, a braided segment and a displacement segment. In some embodiments, the braided segment forms the dynamically expandable distal end. In some embodiments, the probe actuator moves the displacement segment relative to the core segment to expand the braided segment.

In some embodiments, a proximal end of the core segment is coupled to the probe actuator and a distal end of the core segment is coupled to a distal end of the braided segment, while a proximal end of the displacement segment is coupled to the probe actuator and a distal end of the displacement segment is coupled to a proximal end of the braided segment. In some embodiments, the probe actuator expands the dynamically expandable distal end by moving the displacement segment relative to the core segment. In some embodiments, the probe actuator expands the dynamically expandable distal end by moving the proximal end of the displacement segment distally relative to the proximal end of the core segment. In some embodiments, the probe actuator expands the dynamically expandable distal end by moving the proximal end of the displacement segment distally and moving the proximal end of the core segment proximally.

In some embodiments, the probe actuator expands the dynamically expandable distal end via rotational or linear motion. In some embodiments, the dynamically expandable distal end comprises a braided segment and the probe actuator expands the dynamically expandable distal end by shortening the braided segment. In some embodiments, the probe actuator contracts the dynamically expandable distal end by lengthening the braided segment.

In some embodiments, a vascular access system may include a catheter adapter from which a catheter extends distally and a probe assembly that is configured to couple to the catheter adapter. The probe assembly may include a probe housing, a probe that extends within the probe housing and that has a core segment, a braided segment and a displacement segment, and a probe actuator that is configured to extend the probe distally from the catheter while the braided segment is in a flow-reducing position and to transition the braided segment into an open position after the probe is extended distally from the catheter.

In some embodiments, the braided segment has a distal end that is coupled to the core segment and a proximal end that is coupled to the displacement segment. In some embodiments, the core segment has a proximal end that is coupled to the probe actuator and the displacement segment has a proximal end that is coupled to the probe actuator. In some embodiments, the probe actuator transitions the braided segment into the open position by moving the displacement segment relative to the core segment. In some embodiments, the braided segment is transitioned into the open position by shortening the braided segment.

In some embodiments, a method for accessing a vasculature may include accessing a probe assembly that is coupled to a catheter adapter having a catheter that is inserted into a patient's vasculature. The probe assembly may include a probe housing, a probe that extends within the probe housing and a probe actuator. The probe actuator can be slid in a distal direction to cause the distal end of the probe to extend distally from the catheter. After the distal end of the probe extends distally from the catheter, the probe actuator can be manipulated to cause the distal end of the probe to expand.

In some embodiments, the probe has a core segment, a braided segment and a displacement segment. In some embodiments, manipulating the probe actuator causes the displacement segment to move relative to the core segment. In some embodiments, the braided segment has a distal end that is coupled to the core segment and a proximal end that is coupled to the displacement segment.

It is to be understood that both the foregoing general description and the following detailed description are examples and explanatory and are not restrictive of the invention, as claimed. It should be understood that the various embodiments are not limited to the arrangements and instrumentality shown in the drawings. It should also be understood that the embodiments may be combined, or that other embodiments may be utilized and that structural changes, unless so claimed, may be made without departing from the scope of the various embodiments of the present invention. The following detailed description is, therefore, not to be taken in a limiting sense.

### BRIEF DESCRIPTION OF THE DRAWINGS

Example embodiments will be described and explained with additional specificity and detail through the use of the accompanying drawings in which:

FIG. 1 is a perspective view of a vascular access system that includes a dynamically expandable probe configured in accordance with one or more embodiments;

FIG. 2A is a cross-sectional view of a dynamically expandable probe in flow-reducing and open configurations respectively in accordance with one or more embodiments;

FIG. 2B is a cross-sectional view of a dynamically expandable probe in flow-reducing and open configurations respectively in accordance with one or more embodiments;

FIG. 3A is a side view of a dynamically expandable probe that is configured in accordance with one or more embodiments;

FIG. 3B is a side view of a dynamically expandable probe that is configured in accordance with one or more embodiments;

FIG. 3C is a perspective view of a vascular access system configured in accordance with one or more embodiments;

FIG. 4A is a perspective side view of a vascular access system that includes a dynamically expandable probe configured in accordance with one or more embodiments;

FIG. 4B is close-up partial view of FIG. 4A;

FIG. 5A is a perspective sectional view of a braided segment of a dynamically expandable probe that may be used in one or more embodiments; and

FIG. 5B is a perspective sectional view of a braided segment of a dynamically expandable probe that may be used in one or more embodiments.

### DETAILED DESCRIPTION

A vascular access system that may be employed in some embodiments may include a catheter adapter from which a catheter distally extends and one or more ports or connectors for attaching other devices to the catheter adapter. Such devices may be attached to the catheter adapter before, during or after insertion of the catheter into a patient's vasculature and can include a needle assembly, a blood collection set, an infusion assembly, any embodiment of a probe assembly described herein, etc. Accordingly, embodiments of the present disclosure should not be limited to any particular configuration of a vascular access system or to the specific examples of vascular access systems used herein.

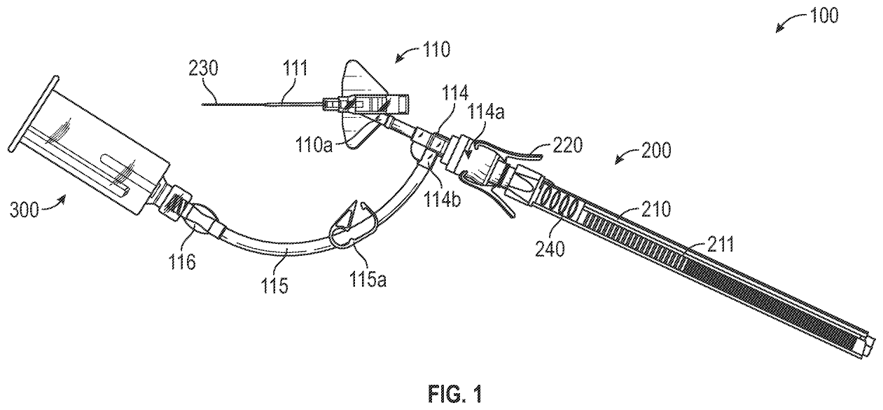

FIG. 1 provides an example of a vascular access system 100 that is configured in accordance with some embodiments of the present disclosure. Vascular access system 100 includes a catheter adapter 110 (or other vascular access device) from which a catheter 111 extends distally. Although not shown, a needle assembly may oftentimes be secured to catheter adapter 110 and may be employed to insert catheter 111 into a patient's vasculature and subsequently detached from catheter adapter 110. Vascular access system 100 also includes an adapter 114 that is connected to a side port 110a of catheter adapter 110.

Vascular access system 100 also includes a probe assembly 200 having a probe housing 210 which can house a probe 230 at least when probe 230 is not extended through catheter 111. A connector 220 can be formed at a distal end of probe housing 210 and can function to connect probe assembly 200 to vascular access system 100 (e.g., via a port 114a of adapter 114 as shown in FIG. 1). In other embodiments, however, probe housing 210 may be integrated into adapter 114 or another component of catheter adapter 110. In other words, how a probe assembly is connected to a catheter adapter is not essential to embodiments of the present disclosure.

Probe assembly 200 may also include a probe actuator 240 that extends out from probe housing 210 and slides along a channel 211 formed in probe housing 210. Probe actuator 240 allows a clinician to move probe 230 relative to catheter 111 by sliding probe actuator 240 along the length of probe housing 210 within channel 211. As described in detail below, a probe assembly configured in accordance with embodiments of the present disclosure may include a dynamically expandable probe that a clinician can selectively control via probe actuator 240. Through this selective control, the clinician can adjust the outer diameter and the permeability of the distal end of the probe. Probe assembly 200, as depicted in FIG. 1, is only one example of how a probe assembly may be configured in embodiments of the present disclosure.

Vascular access system 100 also includes extension tubing 115 that is coupled at one end to a port 114b of adapter 114 and includes an adapter 116 at the opposing end. A blood collection set 300 may be coupled to or integrated with adapter 116. A clamp 115a may be positioned around extension tubing 115 to selectively block the flow of fluid through the extension tubing. FIG. 1 shows probe actuator 240 in its distal-most position and therefore the distal end of probe 230 is advanced distally out from the distal opening of catheter 111

A probe having a dynamically expandable distal end may be used to bypass, move or remove an occlusion that may have formed around the distal opening of a catheter and/or to reposition the catheter such as when its distal opening may be occluded by a vessel wall or other vasculature structure. For example, after inserting catheter 111 into the patient's vasculature but prior to advancing probe 230 through catheter 111, a thrombus could form around catheter 111's opening and prevent blood or fluid from flowing through catheter 111. In such a case, probe actuator 240 could be moved into the distal-most position to advance probe 230, and particularly its dynamically expandable distal end, distally out through the distal opening of catheter 111. The advancement of probe 230 through the distal opening would bypass, move or remove any occlusion that may have formed. Also, the dynamically expandable distal end would allow the probe to remain in a flow-reducing position while it passes through or by the thrombus and then transitioned into an open position to facilitate the collection of blood or the injection of fluid while probe 230 is positioned in and extends distally out through catheter 111's distal opening. This dynamic expandability can minimize the likelihood that probe 230 may become occluded.

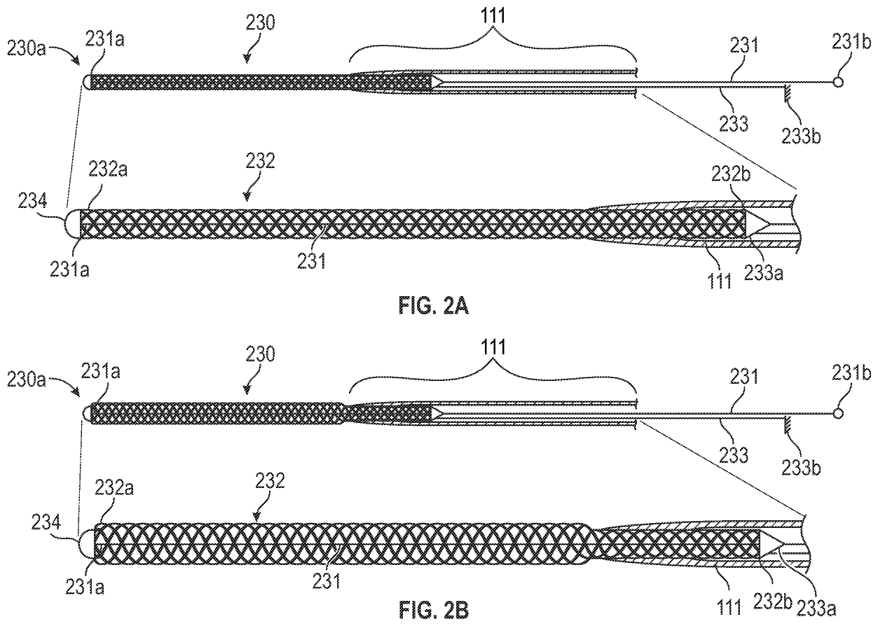

FIGS. 2A and 2B provide examples of probe 230 in a flow-reducing (or unexpanded) position and an open (or expanded) position respectively when the probe is extended distally out from a catheter 111. As shown, probe 230 includes a core segment 231, a braided segment 232 and a displacement segment 233. In some embodiments, core segment 231 and displacement segment 233 may be in the form of two separate wires that couple to opposing ends of braided segment 232. In other embodiments, core segment 231 and displacement segment 233 may be in the form of a single wire having ends that couple to opposing ends of braided segment 232. In some embodiments, core segment 231 and displacement segment 233 may be made from a variety of materials including, for example, stainless steel, nickel titanium alloys such as Nitinol, and nickel, titanium and cobalt (NiTiCo) alloys. In some embodiments, braided segment 232 may be formed of stainless steel, nickel titanium alloys such as Nitinol, NiTiCo alloys, fibers, polymers, elastomers or any other suitable material that can be formed into a braid.

In the depicted embodiment, a distal end 231a of core segment 231 may be connected to a distal end 232a of braided segment 232 at a distal end 230a of probe 230. In some embodiments, distal end 230a can include an atraumatic tip 234. Core segment 231 may be substantially straight with its distal portion extending within braided segment 232 and its proximal end 231b extending proximally to probe actuator 240 (not shown in FIGS. 2A and 2B). In some embodiments, the length of braided segment 232 may be configured to cause proximal end 232b of braided segment 232 to be positioned within catheter 111 when probe 230 is in its distal-most position. For example, FIGS. 2A and 2B can both represent an embodiment where probe 230 is in its distal-most position and proximal end 232b of braided segment 232 remains within catheter 111. In other embodiments, however, braided segment 232 may be positioned entirely outside of catheter 111 when probe 230 is in its distal-most position.

Displacement segment 233 can include a distal end 233a that is coupled to proximal end 232b of braided segment 232 and a proximal end 233b that extends proximally to probe actuator 240. In embodiments where core segment 231 and displacement segment 233 are formed by the same wire, proximal ends 231b and 233b could be viewed as a point on that same wire. Displacement segment 233 can be configured to move distally and proximally relative to core segment 231 to thereby change the distance between distal end 232a and proximal end 232b of braided segment 232. In other words, core segment 231 and displacement segment 233 are configured to change the length of braided segment 232 which in turn changes the outside diameter of braided segment 232. In particular, FIG. 2A shows braided segment 232 in a lengthened state which causes its outside diameter to be reduced such that the openings (or spaces) between braids are minimized. Braided segment 232 can be moved into this flow-reducing position by moving distal end 233a of displacement segment 233 away from distal end 231a of core segment 231. In contrast, FIG. 2B shows braided segment 232 in a shortened state which causes its outside diameter to be expanded such that the openings between braids are increased. Braided segment 232 can be moved into this open position by moving distal end 233a of displacement segment 233 towards distal end 231a of core segment 231. In addition to increasing the size of the openings, the expansion of braided segment 232 can also cause a thrombus to be pushed outwardly away from the opening of catheter 111.

When probe 230 is in the flow-reducing (or unexpanded) position, such as is represented in FIG. 2A, the outside diameter of braided segment 232 may substantially match or be nominally less than the diameter of catheter 111's distal opening. Accordingly, braided segment 232 can be advanced or withdrawn through the distal opening of catheter 111 to position braided segment 232 in a desired location within a patient's vasculature. A clinician could then attempt to draw blood through probe 230 while it remains in the flow-reducing position. If the blood collection is not successful, or if blood flow is insufficient, the clinician may cause relative displacement between core segment 231 and displacement segment 233 to thereby transition probe into (or towards) the open position such as is represented in FIG. 2B. In this open position, the expansion of braided segment 232 will enable blood to flow more freely through braided segment 232 and into catheter 111. In some embodiments, catheter 111 may include additional openings (e.g., on the sides of catheter 111 towards the distal opening) through which blood may also be collected.

Notably, if a thrombus is present on, over or near the distal opening of catheter 111 when probe 230 is advanced distally through the distal opening, braided segment 232 can function to move the thrombus away from the distal opening. Given that braided segment 232 can be in the unexpanded position while moving or otherwise contacting the thrombus, the smaller openings formed between the braids will reduce the likelihood that the thrombus will pass into the interior of probe 230 or catheter 111. Also, because the openings extend along and around the length of braided segment 232, braided segment 232 can enhance blood or fluid flow even if the thrombus blocks some of the openings.

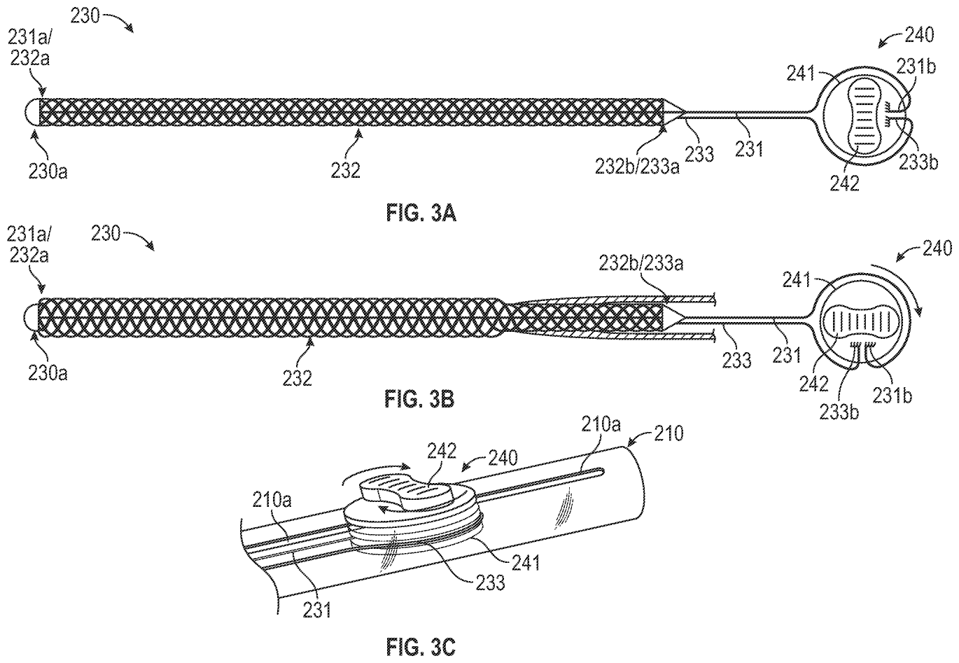

Probe assembly 200 can be configured in a variety of ways to enable this dynamic expansion of braided segment 232. FIGS. 3A-3C provide one example. As shown, proximal ends 231b and 233b of core segment 231 and displacement segment 233 respectively can be connected to a base portion 241 of probe actuator 240. Base portion 241 may have a rounded shape with core segment 231 and displacement segment 233 being routed around opposing sides of the rounded shape. Probe actuator 240 may also include a tab 242 to enable a clinician to rotate probe actuator 240. As represented in FIG. 3A, probe actuator 240 could be configured to cause probe 230 to be in the flow-reducing position by default. For example, braided segment 232 could be configured to not be expanded absent an external force, or probe actuator 240 could otherwise be biased into the position shown in FIG. 3A.

As represented in FIG. 3B, the rotation of probe actuator 240 can pull core segment 231 proximally relative to displacement segment 233 thereby shortening braided segment 232 and causing its outside diameter to expand. More specifically, the rotation pulls distal end 232a of braided segment 232 proximally while pushing proximal end 232b of braided segment 232 distally (or otherwise causes relative movement of distal end 232a towards proximal end 232b).

As represented in FIG. 3C, probe actuator 240 can be configured to slide distally and proximally within channel 211 to thereby move distal end 230a of probe 230 distally and proximally respectively relative to catheter 111. For example, probe actuator 240 could be moved distally to extend probe 230 from catheter 111 and moved proximally to retract probe 230 into catheter 111. Once probe 230 is moved into a desired position (e.g., once extended from catheter 111), probe actuator 240 can be rotated to dynamically expand and contract probe 230 as described above.

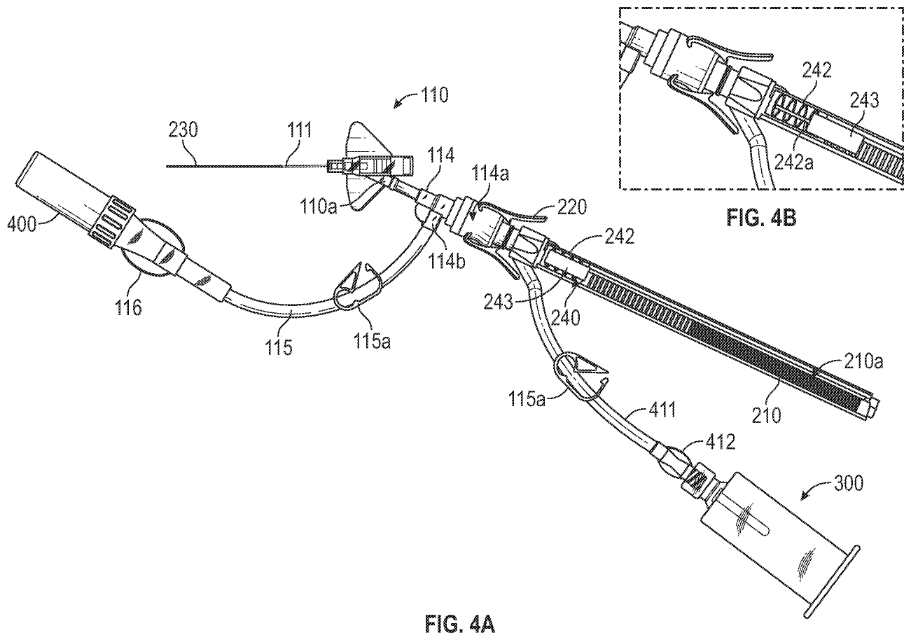

FIGS. 4A and 4B, which are similar to FIG. 1, provide another example of how probe actuator 240 may be configured to enable a clinician to dynamically expand probe 230. In FIGS. 4A and 4B, probe actuator 240 is in its distal-most position, and therefore, probe 230 extends distally out from catheter 111. Although not visible, in FIG. 4A, probe 230 is in the flow-reducing position, whereas in FIG. 4B, probe 230 is in the open position. Probe actuator 240 can include a sliding tab 243 that can be coupled to proximal end 231b of core segment 231. With sliding tab 243 in the distal position shown in FIG. 4A, core segment 231 will not be pulled proximally relative to displacement segment 233 thereby causing probe 230 to be in the flow-reducing (unexpanded) position. In contrast, when sliding tab 243 is slid proximally relative to tab 242 as shown in FIG. 4B, it will pull core segment 231 proximally thereby shortening the distance between distal end 232a and proximal end 232b of braided segment 232. As stated above, in some embodiments, this proximal movement of core segment 231 alone may transition probe 230 from the flow-reducing position to or towards the open position. However, in some embodiments, probe actuator 240 may also include a second sliding tab (e.g., on the opposite side of probe actuator 240 from sliding tab 243) to which proximal end 233b of displacement segment 233 is connected. In such cases, this second sliding tab can be moved distally to move proximal end 232b distally while tab 243 is moved proximally to move distal end 232a proximally to thereby transition probe 230 from the flow-reducing position to or towards the open position. Accordingly, a variety of configurations and techniques can be employed to cause relative movement between distal end 232a and proximal end 232b of braided segment 232.

FIGS. 4A and 4B also represent embodiments where probe assembly 200 forms a fluid pathway. For example, probe housing 210 is shown as including a port 410 from which extension tubing 411 extends. Blood collection set 300 can be connected to an adapter 412 at the end of extension tubing 411. In such embodiments, a needle-less connector 400 or other device can be coupled to adapter 116.

Probe 230 has been described as having two positions: an open position and a flow-reducing position. However, in any embodiment, probe 230 could have a flow-reducing position and variable open positions. For example, in the embodiment shown in FIGS. 3A-3C, probe actuator 240 could be rotated to open probe 230 to a desired amount. Likewise, in the embodiment shown in FIGS. 4A and 4B, sliding tab 243 could be slid to open probe 230 to a desired amount. Accordingly, probe actuator 240 could be configured in a wide variety of ways to cause probe 230 to have a single open position or many open positions. In some embodiments, this variability in the permeability of braided segment 232 can enable the clinician to control the rate of fluid or blood flow through probe 230.

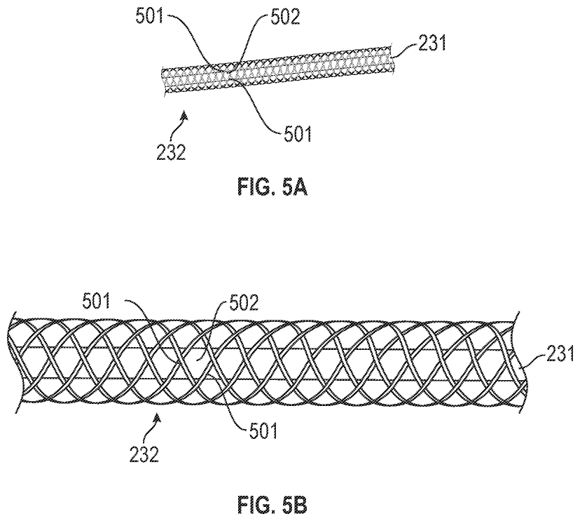

FIGS. 5A and 5B provide a more detailed example of how braided segment 232 enables the permeability of probe 230 to be controlled in one or more embodiments. In FIG. 5A, braided segment 232 is in the unexpanded configuration and therefore the strands 501 that form braided segment 232 create smaller openings 502. In contrast, in FIG. 5B, braided segment 232 is in the expanded configuration and therefore the strands 501 create larger openings 502.

In some embodiments, probe 230 can be configured to enable braided segment 232 to be constricted to the point where it contacts core segment 231. For example, distal end 233a of displacement segment 233 can be pulled sufficiently away from distal end 231a of core segment 231 (and/or vice versa) to cause braid section 232 to squeeze core segment 231. In such cases, this may be viewed as a closed position in which probe 230 prevents or severely limits the amount of blood that may flow into catheter 111 or fluid that may flow out of catheter 111. Accordingly, in some embodiments, a probe may be transitionable from a closed position to an increasing degree of open positions.

In some embodiments, a variety of braid patterns may be used to form braided segment 232. For example, the strands 501 that form braided segment 232 can have a varying angle and/or spacing to thereby cause the size and/or shape of openings 502 to vary. In some embodiments, strands 501 can be configured to cause openings 502 to have any of a variety of shapes including diamond, trapezoidal, triangular, rectangular, square, oval, oblong, etc. In some embodiments, the effective width of openings 502 may be between 0.000 inches and 0.015 inches. In some embodiments, the effective width of openings 502 may exceed 0.015 inches.

In summary, a probe configured in accordance with embodiments of the present disclosure can be dynamically expanded to thereby increase the permeability of the probe and to move thrombus away from the catheter's opening. The expansion of the probe can also cause the catheter's opening to be positioned towards the center of the patient's vasculature thereby reducing the risk that the opening will be occluded by the vasculature wall.

In a typical use case, the probe can be in the flow-reducing position while it is advanced distally from a catheter to a desired location within the patient's vasculature. By advancing the probe in the flow-reducing state as opposed to in the open state, the probe can provide greater stiffness to the catheter. Once positioned, the clinician can open the probe to better collect a blood sample and/or to inject a fluid through the catheter. By inserting the probe in the flow-reducing position, the risk of a thrombus occluding the probe is minimized. Also, the clinician can control the degree to which the probe is opened (or more particularly, the size of openings between braids) to thereby minimize the likelihood of a thrombus entering the probe while drawing blood.

All examples and conditional language recited herein are intended for pedagogical objects to aid the reader in understanding the invention and the concepts contributed by the inventor to furthering the art, and are to be construed as being without limitation to such specifically recited examples and conditions. Although embodiments of the present inventions have been described in detail, it should be understood that the various changes, substitutions, and alterations could be made hereto without departing from the spirit and scope of the invention.

What is claimed is:

1. A probe assembly comprising:
a probe housing;
a probe that extends within the probe housing, the probe having a dynamically expandable distal end; and
a probe actuator that is configured to advance the probe from a proximal position to a distal position, the probe actuator also being configured to expand the dynamically expandable distal end.

2. The probe assembly of claim 1, wherein the probe comprises a core segment, a braided segment and a displacement segment.

3. The probe assembly of claim 2, wherein the braided segment forms the dynamically expandable distal end.

4. The probe assembly of claim 3, wherein the probe actuator moves the displacement segment relative to the core segment to expand the braided segment.

5. The probe assembly of claim 2, wherein:
a proximal end of the core segment is coupled to the probe actuator and a distal end of the core segment is coupled to a distal end of the braided segment; and
a proximal end of the displacement segment is coupled to the probe actuator and a distal end of the displacement segment is coupled to a proximal end of the braided segment.

6. The probe assembly of claim 5, wherein the probe actuator expands the dynamically expandable distal end by moving the displacement segment relative to the core segment.

7. The probe assembly of claim 6, wherein the probe actuator expands the dynamically expandable distal end by moving the proximal end of the displacement segment distally relative to the proximal end of the core segment.

8. The probe assembly of claim 6, wherein the probe actuator expands the dynamically expandable distal end by moving the proximal end of the displacement segment distally and moving the proximal end of the core segment proximally.

9. The probe assembly of claim 1, wherein the probe actuator expands the dynamically expandable distal end via rotational or linear motion.

10. The probe assembly of claim 1, wherein the dynamically expandable distal end comprises a braided segment, and wherein the probe actuator expands the dynamically expandable distal end by shortening the braided segment.

11. The probe assembly of claim 10, wherein the probe actuator contracts the dynamically expandable distal end by lengthening the braided segment.

12. A vascular access system comprising:
a catheter adapter from which a catheter extends distally; and
a probe assembly that is configured to couple to the catheter adapter, the probe assembly comprising:
a probe housing;
a probe that extends within the probe housing, the probe having a core segment, a braided segment and a displacement segment; and
a probe actuator that is configured to extend the probe distally from the catheter while the braided segment is in a flow-reducing position and to transition the braided segment into an open position after the probe is extended distally from the catheter.

13. The vascular access system of claim 12, wherein the braided segment has a distal end that is coupled to the core segment and a proximal end that is coupled to the displacement segment.

14. The vascular access system of claim 13, wherein the core segment has a proximal end that is coupled to the probe actuator and the displacement segment has a proximal end that is coupled to the probe actuator.

15. The vascular access system of claim 14, wherein the probe actuator transitions the braided segment into the open position by moving the displacement segment relative to the core segment.

16. The vascular access system of claim 12, wherein transitioning the braided segment into the open position comprises shortening the braided segment.

17. A method for accessing a vasculature comprising:
accessing a probe assembly that is coupled to a catheter adapter having a catheter that is inserted into a patient's vasculature, the probe assembly comprising a probe housing, a probe that extends within the probe housing and a probe actuator;
sliding the probe actuator in a distal direction to cause a distal end of the probe to extend distally from the catheter; and
after the distal end of the probe extends distally from the catheter, manipulating the probe actuator to cause the distal end of the probe to expand.

18. The method of claim 17, wherein the probe has a core segment, a braided segment and a displacement segment.

19. The method of claim 18, wherein manipulating the probe actuator to cause the distal end of the probe to expand comprises manipulating the probe actuator to cause the displacement segment to move relative to the core segment.

20. The method of claim 19, wherein the braided segment has a distal end that is coupled to the core segment and a proximal end that is coupled to the displacement segment.
